(19) United States
(12) Patent Application Publication
(10) Pub. No.: US 2021/0154499 A1
(43) Pub. Date: May 27, 2021

(54) DEVICE AND METHODS FOR TREATING NEUROLOGICAL DISORDERS AND BRAIN CONDITIONS

(71) Applicant: Liminal Sciences, Inc., Guilford, CT (US)

(72) Inventors: Kamyar Firouzi, San Jose, CA (US); Mohammad Moghadamfalahi, San Jose, CA (US)

(73) Assignee: Limited Sciences, Inc., Guilford, CT (US)

(21) Appl. No.: 17/103,612

(22) Filed: Nov. 24, 2020

Related U.S. Application Data
(60) Provisional application No. 62/940,433, filed on Nov. 26, 2019.

(51) Int. Cl. A61N 7/00; A61B 5/00; A61B 5/055; G16H 20/40; G16H 50/50; G16H 50/20

(52) U.S. Cl. CPC A61N 7/00; A61B 5/0036; A61B 5/055; A61N 2007/0078; G16H 50/50; G16H 50/20; A61N 2007/0026; G16H 20/40

(57) ABSTRACT

In some aspects, a device comprises a substrate and at least one CMUT located on or in the substrate that provides ultrasound radiation to a brain of a patient. In some aspects, a method of guiding ultrasound radiation in the brain of a patient comprises receiving as a first input patient scan data, receiving as a second input information regarding configuration and/or properties of ultrasound transmitters adapted to transmit to the brain the ultrasound radiation, processing at least one of the first and second inputs and feeding the processed at least one of the first and second inputs into a physical acoustics model, and based on an output of the physical acoustics model and acquired data from the brain of the patient, generating an instruction to transmit to the brain of the patient the ultrasound radiation.

### CROSS REFERENCE TO RELATED APPLICATIONS

This application claims priority under 35 U.S.C. § 119(e) to U.S. Provisional Application Ser. No. 62/940,433, titled “DEVICE AND METHODS FOR TREATING NEUROLOGICAL DISORDERS AND BRAIN CONDITIONS,” filed Nov. 26, 2019, which is hereby incorporated herein by reference in its entirety.

### BACKGROUND

Neurological disorders affecting brain health constitute a significant portion of the global burden of disease. Such disorders can include epilepsy, Alzheimer's disease, and Parkinson's disease. For example, about 65 million people worldwide suffer from epilepsy. In the developing world, onset is more common in older children and young adults, due to differences in the frequency of the underlying causes. Nearly 80% of cases occur in the developing world. In the developed world, onset of new cases occurs most frequently in babies and the elderly. The United States itself has about 3.4 million people suffering from epilepsy with an estimated $15 billion economic impact. These patients suffer from symptoms such as recurrent seizures, which are episodes of excessive and synchronized neural activity in the brain. In many areas of the world, those with epilepsy either have restrictions placed on their ability to drive or are not permitted to drive until they are free of seizures for a specific length of time.

### SUMMARY

The inventors have appreciated that conventional techniques to transmit ultrasound signals into the brain to treat neurological disorders are implemented by means of a large-aperture spherical transducer consisting of a very large number of single element transducers transmitting ultrasound beams through the skull. Some technologies rely on the use of an array of transducers that are placed in a helmet. The inventors have discovered that the geometric focus of these transducers limits the treatment envelope to the center of the brain, whereas the majority of neurological disorders and cancers, especially metastases, occur along or originate in the periphery of the brain. Still other approaches rely on magnetic resonance guidance in real time, which is very bulky and expensive. The inventors have discovered other problems with these approaches, including self-heating and inefficiency in the transmission of the ultrasound signals.

To address these shortcomings, the inventors have developed a novel device and methods for introducing and guiding ultrasound signals into the brain in the form of focused, unfocused, and/or divergent beams. These ultrasound beams may be used for therapy or neuro-modulation, in a manner that may be non-invasive or minimally invasive (e.g., ultrasound transmitter placed under the scalp), wired or wireless, and/or with the capability of providing continuous or acute therapy. The ultrasound beam may be steered using machine learning or another suitable means. Such ultrasound signals may be used to modulate neural activity, stop seizures, and/or otherwise treat one or more portions of the brain. For example, low intensity focused ultrasound (LIFU) signals may be used to excite, or inhibit, neural activity in the brain, e.g., to mitigate a seizure or another brain condition. The described device and methods may accordingly be used for treating brain conditions and/or neurological disorders. The neurological disorders include but are not limited to epileptic seizures, depression, Alzheimer's disease, Parkinson's disease, and other disorders. The brain conditions include but are not limited to brain tumors, stroke, traumatic brain injury, vasospasm, and other conditions.

In some aspects, a device comprises a substrate and at least one capacitive micromachined ultrasonic transducer (CMUT) located on or in the substrate that provides ultrasound radiation to a brain of a patient.

In some embodiments, the substrate is flexible.

In some embodiments, the substrate is made from a printed circuit board (PCB).

In some embodiments, the at least one CMUT includes an array of a plurality of CMUTs.

In some embodiments, the substrate is embedded in or on a cap intended to be worn on a scalp of the patient.

In some embodiments, the at least one CMUT is powered and/or driven wirelessly.

In some embodiments, the ultrasound radiation is guided within the brain through a computer implemented simulation model.

In some embodiments, the computer implemented simulation model includes a machine learning model.

In some embodiments, the computer implemented simulation model includes as an input a scan of the brain of the patient.

In some embodiments, the ultrasound radiation is guided within the brain of the patient through magnetic resonance imaging (MRI) monitoring.

In some aspects, a wearable or implantable device for disposal on a scalp of a patient comprises a substrate and at least one capacitive micromachined ultrasonic transducer (CMUT) located on or in the substrate that provides ultrasound radiation to a brain of the patient.

In some aspects, a method of guiding ultrasound radiation in the brain of a patient comprises receiving as a first input patient scan data, receiving as a second input information regarding configuration and/or properties of one or more ultrasound transmitters adapted to transmit to the brain the ultrasound radiation, processing at least one of the first and second inputs and feeding the processed at least one of the first and second inputs into a physical acoustics model, and based on an output of the physical acoustics model and acquired data from the brain of the patient, generating an instruction to transmit to the brain of the patient the ultrasound radiation.

In some embodiments, the method further comprises feeding the output of the physical acoustics model and the acquired data from the brain of the patient into a machine learning model, and based on an output of the machine learning model, generating the instruction to transmit to the brain of the patient the ultrasound radiation.

In some embodiments, the configuration includes a spatial arrangement of the one or more ultrasound transmitters.

In some embodiments, the properties include at least one of sound signal speeds, elasticity, and/or density.

In some embodiments, the physical acoustics model employs at least one of linear acoustics, non-linear acoustics, electrodynamics, and/or non-linear continuums.

In some embodiments, the acquired data from the brain of the patient fed into the machine learning model includes at least one of a frequency response, an impulse/transient response, and/or a distribution of acoustic modes.

In some embodiments, the output of the machine learning model includes at least one of frequency, amplitude, acoustic beam profile, temperature elevation or reduction, and/or radiation force.

In some embodiments, the machine learning model comprises a convolutional neural network.

In some embodiments, the method further includes building the machine learning model and/or training with data the machine learning model.

In some embodiments, the method further comprises feeding the output of the physical acoustics model and updated data acquired from the brain of the patient into the machine learning model.

In some embodiments, the method further comprises generating an updated instruction to transmit to the brain of the patient the ultrasound radiation.

While some aspects and/or embodiments described herein are described with respect to epilepsy-related applications, these aspects and/or embodiments may be equally applicable to monitoring and/or treating symptoms for any suitable neurological disorder or brain condition. Any limitations of the embodiments described herein are limitations only of those embodiments, and are not limitations of any other embodiments described herein.

### BRIEF DESCRIPTION OF THE DRAWINGS

Various aspects and embodiments will be described with reference to the following figures. The figures are not necessarily drawn to scale.

FIG. 1 shows an illustrative embodiment of a device and a hub for treating a neurological disorder, in accordance with some embodiments of the technology described herein.

FIG. 2 shows an illustrative embodiment of a device for treating a neurological disorder, in accordance with some embodiments of the technology described herein.

FIG. 3 shows illustrative embodiments of a device for treating a neurological disorder, in accordance with some embodiments of the technology described herein.

FIG. 4 shows illustrative simulations of focusing performance of a capacitive micromachined ultrasonic transducer (CMUT) array included in a device for treating a neurological disorder, in accordance with some embodiments of the technology described herein.

FIG. 5 shows an illustration of a CMUT cell, in accordance with some embodiments of the technology described herein.

FIG. 6 shows an overview of an illustrative algorithm for generating a machine learning model to be used in treating a neurological disorder, in accordance with some embodiments of the technology described herein.

FIG. 7 shows an illustrative flow diagram for a process for constructing and deploying an algorithm, e.g., as shown in FIG. 6, in accordance with some embodiments of the technology described herein.

FIG. 8 shows an illustrative flow diagram for guiding ultrasound radiation in a device for treating a neurological disorder, in accordance with some embodiments of the technology described herein.

FIG. 9 shows a convolutional neural network that may be used in conjunction with a device for treating a neurological disorder. in accordance with some embodiments of the technology described herein.

FIG. 10 shows a block diagram of an illustrative computer system that may be used in implementing some embodiments of the technology described herein.

### DETAILED DESCRIPTION

In some aspects, the inventors have developed a novel device and methods for introducing and guiding ultrasound signals into the brain in the form of focused, unfocused, and/or divergent beams. The ultrasound beam may be used for therapy or neuro-modulation, in a manner that may be non-invasive or minimally invasive (e.g., ultrasound transmitter placed under the scalp), wired or wireless, and/or with the capability of providing continuous or acute therapy. The ultrasound beam may be steered using machine learning or another suitable means. Such ultrasound signals may be used to modulate neural activity, stop seizures, and/or otherwise treat one or more portions of the brain.

Accordingly, the described device and methods may be used for treating brain conditions and/or neurological disorders. The neurological disorders include but are not limited to epileptic seizures, depression, Alzheimer's disease, Parkinson's disease, and other disorders. The brain conditions include but are not limited to brain tumors, stroke, traumatic brain injury, vasospasm, and other conditions.

In some embodiments, the described device is compact, can be wearable or implantable, and is in a form factor comfortable to a human being. The device may have a compact form factor with wired or wireless charging and monitoring capability. The device may be miniaturized as appropriate for certain applications (e.g., for children) and/or fabricated on a flexible printed circuit board (PCB) that can conform to a human head curvature and geometry. The device may be integrated with application specific integrated circuits (ASICs) and/or electronics on a single chip. The device may be able to transmit and receive data through wires and/or wirelessly. In some embodiments, ultrasound signals from the device to the brain may be guided/navigated via either in-silico model (e.g., simulation-based and enhanced with machine learning techniques), or external means of monitoring, or a combination of both external and internal methods.

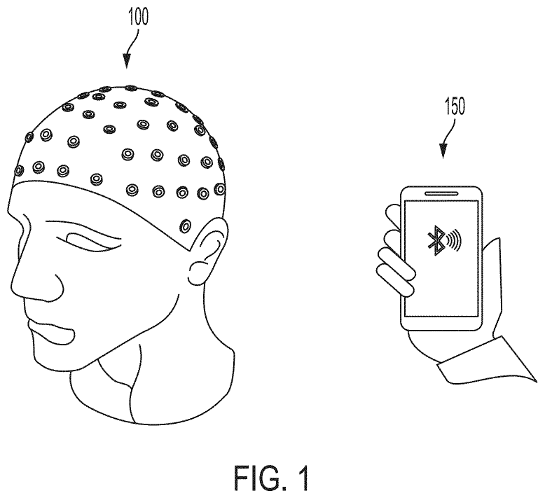

FIG. 1 shows an illustrative embodiment of a device 100 and a hub 150 for treating a neurological disorder, in accordance with some embodiments of the technology described herein. In the illustrated wearable form, the device 100 is integrated into a helmet or a cap. The device can be charged wired or wirelessly and transfer data to a hub 150 that can be worn (e.g., as a watch or a smart phone) or implanted (e.g., a small patch over the neck/arm). In some embodiments, the form factor for the device can be one or several small adhesive patches. In some embodiments, the device can be either wearable or implantable (e.g., under the scalp).

In some embodiments, focused ultrasound energy from the device can be utilized for therapeutic and/or neuro-modulation applications. Brain metastases, the most common malignant brain tumors, occur in up to 40% of patients with cancer. Left untreated, prognosis is abysmal, with a life expectancy of one month. Surgery and radiation are typically combined to treat brain metastases. In order to minimize or avoid the risks of invasive surgery, such as bleeding and infection, and the toxic effects of radiation to the brain, such as decline in learning and memory, alternatives such as the described device are sought for therapeutic and/or neuro-modulation applications. In some embodiments, the described device may use magnetic resonance guided focused ultrasound as a noninvasive means of ablating brain tumors and of increasing delivery of cancer therapeutics through the blood-brain barrier.

The described non-invasive neuro-modulation may be used in treating diseases like stroke, multiple sclerosis, neuropathic pain, migraine, depression, etc. Transcranial Magnetic Stimulation (TMS) is conventionally the most common modality, however, with poor spatial selectivity and penetration depth. The inventors have appreciated that ultrasound neuro-modulation is a competing technique with superior spatial selectivity and penetration depth, and potentially a wider spectrum of applications.

Because neurons in the brain are sensitive to ultrasound, if ultrasound sequences are applied with properties including but not limited to certain carrier frequencies, pulse durations, pulse repetition frequencies, burst durations, and power levels, neurons will become more or less active (e.g., as measured by the rate at which they generate action potentials). The ultrasound transmitter(s) in the described device may be used to send focused ultrasound radiation through the skull and into the brain to selectively activate and/or inhibit groups of neurons. In using ultrasound for neural modulation, ultrasound signals may be transmitted at the scalp through the entire thickness of the skull and through a certain distance of brain tissue (e.g., on the order of 10 cm or less).

In some embodiments, non-limiting fields of application of the described device and methods includes essential tremor, Parkinson's disease, tremor dominant, depression, neuropathic pain, obsessive-compulsive disorder, dyskinesia, Alzheimer' s disease, amyotrophic lateral sclerosis, astrocytoma (SEGA), brain metastases, cancer pain, dementia, dystonia, epilepsy, glioblastoma, Holmes tremor, Huntington's disease, neuroblastoma, pediatric, opening brain blood barrier, painful amputation neuromas, pontine glioma, traumatic brain injury, addiction, cavernomas, hydrocephalus, intracerebral hemorrhage, migraine, multiple sclerosis, seizure, spinal cord injury, tissue ablation, thromboembolic stroke, trigeminal neuralgia, tumor treatment, and/or anorexia.

In some embodiments, the described device includes a substrate and at least one capacitive micromachined ultrasonic transducer (CMUT) located on or in the substrate that provides ultrasound radiation to a brain of a patient. For example, the substrate may be flexible and/or made from a printed circuit board (PCB). In some embodiments, the substrate may be embedded in or on a cap intended to be worn on the scalp of the patient. In some embodiments, the devices described herein may include other type(s) of transducers in place of, or in addition to, CMUTs. For example, the device may include one or more piezoelectric transducers, electro magnetic acoustic transducers (EMATs), piezoelectric micromachined ultrasonic transducers (PMUTs), and/or other suitable types of transducers.

In some embodiments, the device described herein, e.g., device 100, includes a CMUT array that has maximal transmit power in the range of 500 kHz-1 MHz. Conventional CMUT arrays have difficulty operating at such low frequencies. The device can be a single-element, or may be a 1D or 2D array, played out in a geometry such as a grid, a ring, a curved surface, or a similar shape. The array can be populated with many transducer elements such as CMUTs with electronic control over phases and amplitude of elements in groups or individually, to allow electronic steering for creating focused, unfocused, divergent beams, and correcting for beam aberration and attenuation caused by the skull or different brain tissue types. The same device can also perform dual mode imaging and therapy, by switching between the therapeutic and imaging modality, in cases where the images are used to guide the beam. The device can focus at particular locations within the brain, with a resolution of 10s of cubic millimeters, by using beam forming algorithms on the array (by “phasing” the array) or using an acoustic lens on the transducer. To obtain suitable transmission through the skull, carrier frequencies at, near, or below 800 kHz-1 MHz can be used. At such frequencies, significant levels of ultrasound radiation can pass through the skull and reach the brain tissue, which is significantly less attenuating. The brain tissue may defocus the ultrasound beam to some extent, but the inventors have not found this to be an issue in their experiments.

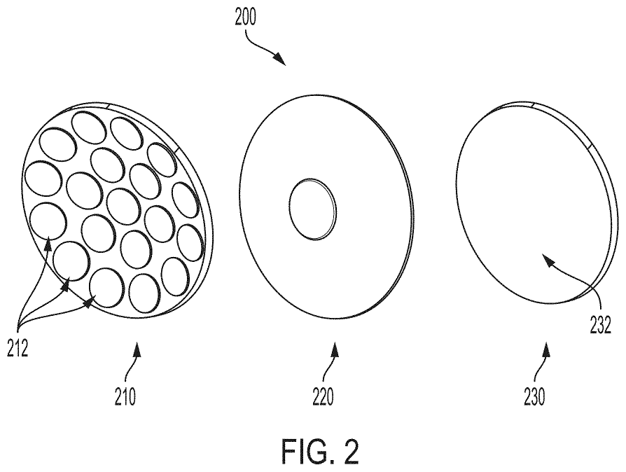

In some embodiments, the described device may include an array of a plurality of CMUTs. The CMUTs may be powered and/or driven wirelessly. FIG. 2 shows an illustrative embodiment of a device 200 for treating a neurological disorder, in accordance with some embodiments of the technology described herein. In particular, FIG. 2 shows different layers of the same transducer, e.g., device 200. The left subfigure 210 shows the elements 212 under the lens 232 (not shown). The right subfigure 230 shows the device 200 with the lens 232 on the elements 212 (not shown). The middle subfigure 220 shows the back side of the device 200. The device 200 has a 2″ diameter of the aperture and 3″ geometrical focus, less than a ⅛″ overall thickness, with a flexible/conforming lens case on the front side, to provide coupling and shape conformation to a person's head. The device and electronics (including application specific integrated circuits (ASICs)) may be integrated on a single chip on a flexible printed circuit board (PCB). As shown in FIG. 2, the device 200 includes a CMUT array with multiple CMUTs (or arrays of transducers) placed on a flexible substrate (e.g., PCB), intended to be placed on the scalp of a person. The device 200 may include wires or an antenna for wireless communication and charging. Driver electronics may be integrated in the CMUT or provided externally thereto.

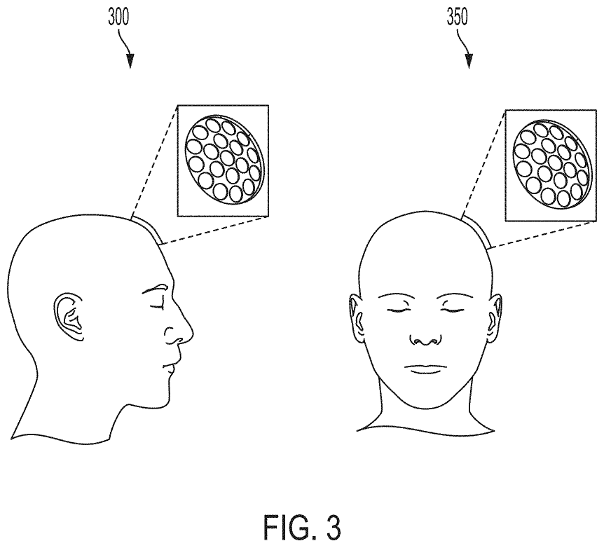

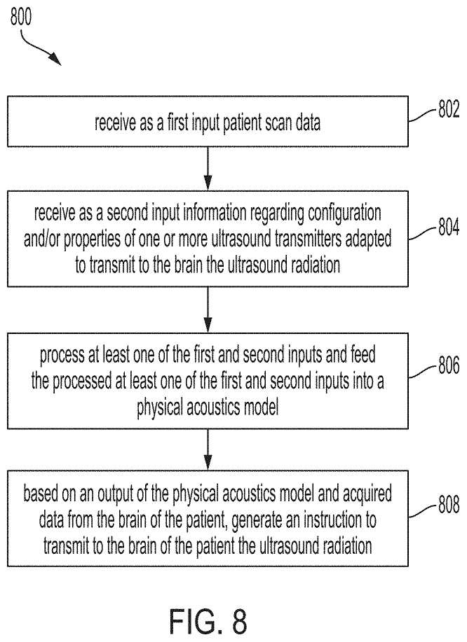

FIG. 3 shows illustrative embodiments 300 and 350 of a device for treating a neurological disorder, in accordance with some embodiments of the technology described herein. The device includes multiple CMUTs located on a flexible cap substrate, e.g., as shown in FIG. 2, and applied to the scalp of a person. Depending on the circumstances, differently sized and shaped caps, and locations for application to the scalp, may be used. The described device may be compact, wearable, and/or in a form factor comfortable to a human being. The device may be configured with wired or wireless charging and monitoring capability. The device may be able to transmit and receive data through wires and/or wirelessly. The ultrasound signals from the device to the brain may be guided/navigated via either in-silico model (e.g., simulation-based and enhanced with machine learning techniques as described with respect to FIG. 8), or external means of monitoring, or a combination of both external and internal methods.

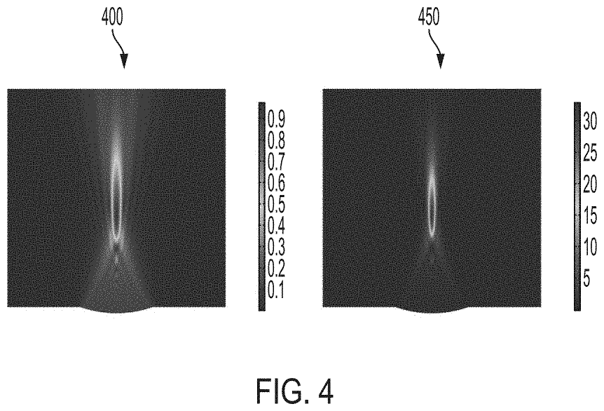

FIG. 4 shows illustrative simulations of focusing performance of a CMUT array included in a device for treating a neurological disorder, in accordance with some embodiments of the technology described herein. The left FIG. 400 shows the pressure beam profile (in MPa). The right FIG. 450 shows the intensity beam profile (in W/cm²). In some embodiments, intensity levels in the approximate range of 3 W/cm² to 30 W/cm² may be effective for neuro-modulation. FIG. 4 shows the described embodiments can achieve these intensity levels for neuro-modulation, or another suitable range of intensity levels suitable for neuro-modulation.

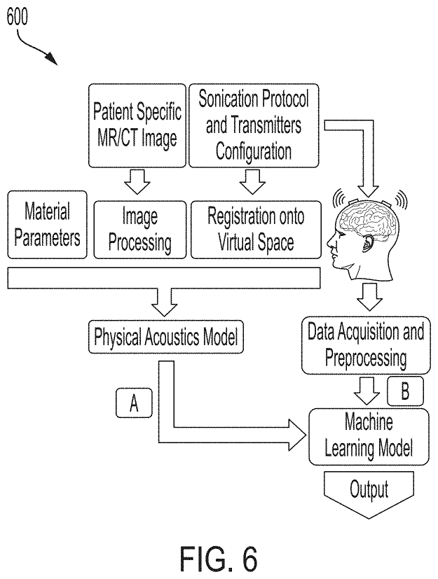

In some embodiments, the ultrasound radiation may be guided within the brain through a computer implemented simulation model, e.g., a machine learning model. The computer implemented simulation model may include as an input a scan of the patient's brain. Additionally or alternatively, the ultrasound radiation may be guided within the brain through magnetic resonance imaging (MRI) monitoring. These aspects of the described device and methods are further described with respect to FIGS. 6-8.

### Transducer Technology

Transducers can be of a variety of types such as piezoelectric, capacitive micromachined ultrasonic transducer (CMUT), electro magnetic acoustic transducer (EMAT), piezoelectric micromachined ultrasonic transducer (PMUT), etc. Material and dimensions determine the bandwidth and sensitivity of the transducer. CMUTs are of particular interest as they can be easily miniaturized even at low frequencies and have superior sensitivity as well as wider bandwidth compared to other types of transducers. CMUTs may be easily miniaturized and integrated with electronics, in particular on flexible substrates. When compared to other types of transducers (e.g., piezoelectric), CMUTs have fewer heating issues because the internal loss in CMUTs may be negligible. Further, compared to other types of transducers, CMUTs may provide better bandwidth and transmit-receive sensitivity, particularly in cases where an array of transducers is used for guiding neuro-modulation treatment.

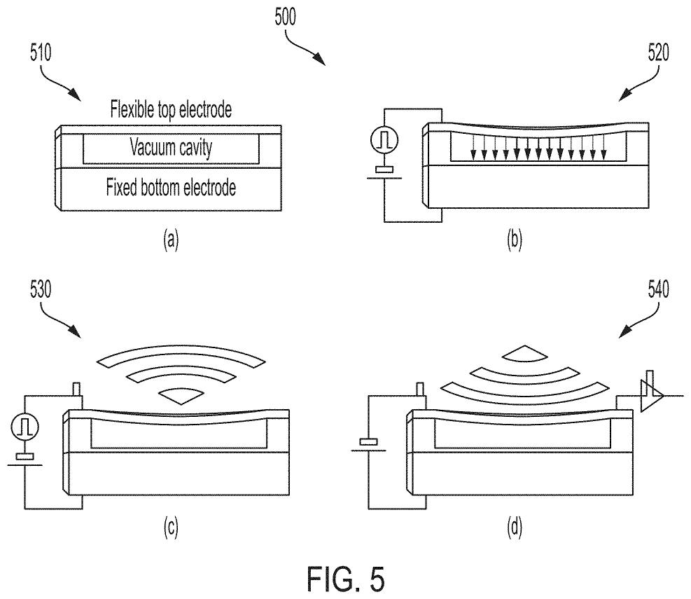

In some embodiments, the CMUT consists of a flexible top plate suspended over a gap, forming a variable capacitor. The displacement of the top plate creates an acoustic pressure in the medium (or vice versa; acoustic pressure In the medium displaces the flexible plate). Transduction is achieved electrostatically, by converting the displacement of the plate to an electric current through modulating the electric field in the gap, in contrast with piezoelectric transducers. The merit of the CMUT derives from having a very large electric field in the cavity of the capacitor, a field of the order of 10\hat{ }8 V/m or higher results in an electro-mechanical coupling coefficient that competes with the best piezoelectric materials. The availability of micro-electro-mechanical-systems (MEMS) technologies makes it possible to realize thin vacuum gaps where such high electric fields can be established with relatively low voltages. Thus, viable devices can be realized and even integrated directly on electronic circuits such as complimentary metal-oxide-semiconductor (CMOS). FIG. 5 shows illustrations 510, 520, 530, and 540 of a CMUT cell (a) without DC bias voltage, and (b) with DC bias voltage, and principle of operation during (c) transmit and (d) receive.

In some embodiments, a further aspect is collapse mode operation of the CMUT. In this mode of operation, the CMUT cells are designed so that part of the top plate is in physical contact with the substrate, yet electrically isolated with a dielectric, during normal operation. The transmit and receive sensitivities of the CMUT are further enhanced thus providing a superior solution for ultrasound transducers. In short, the CMUT is a high electric field device, and if one can control the high electric field from issues like charging and breakdown, then one has an ultrasound transducer with superior bandwidth and sensitivity, amenable for integration with electronics, manufactured using traditional integrated circuits fabrication technologies with all its advantages, and can be made flexible for wrapping around a cylinder or even over human tissue.

### Guiding Ultrasound Radiation

In some embodiments, the ultrasound radiation of the device can be navigated (or guided) either internally, by other external techniques such as magnetic resonance imaging (MRI), or a combination of both. The internal technique to guide the beam may include an in-silico (e.g., simulation-based) method that is enhanced with machine learning, e.g., using a machine learning model as described with respect to FIG. 6. First, before the treatment, a computerized tomography (CT) or magnetic resonance (MR) scan of the patient's head may be acquired (e.g., which may be done during transcranial therapy and monitoring), and a baseline physical-acoustics model may be constructed (e.g., using patient specific structural features and base-line acoustic properties). Through machine learning, the model may be adapted to capture the deviations from the base-line model and “learn” the patient specific parameters. The model can then be utilized during the treatment to guide/navigate the beam.

In some embodiments, machine learning algorithms may be employed in the form of a classification or regression algorithm, which may include one or more sub-components such as convolutional neural networks, recurrent neural networks such as LSTMs and GRUs, linear SVMs, radial basis function SVMs, logistic regression, and various techniques from unsupervised learning such as variational autoencoders (VAE), generative adversarial networks (GANs) which are used to extract relevant features from the raw input data.

In some embodiments, the described technology may be patient-specific, thereby having computations and model-based learning implemented by using the patient's head MR or CT scan. The medical images may be processed and fed into an acoustic solver, which is then used to train the model-based machine learning model. In some embodiments, other techniques to guide the beam may include but are not limited to shear wave elastography, MR thermometry, and neuroimaging techniques, such as functional imaging techniques.

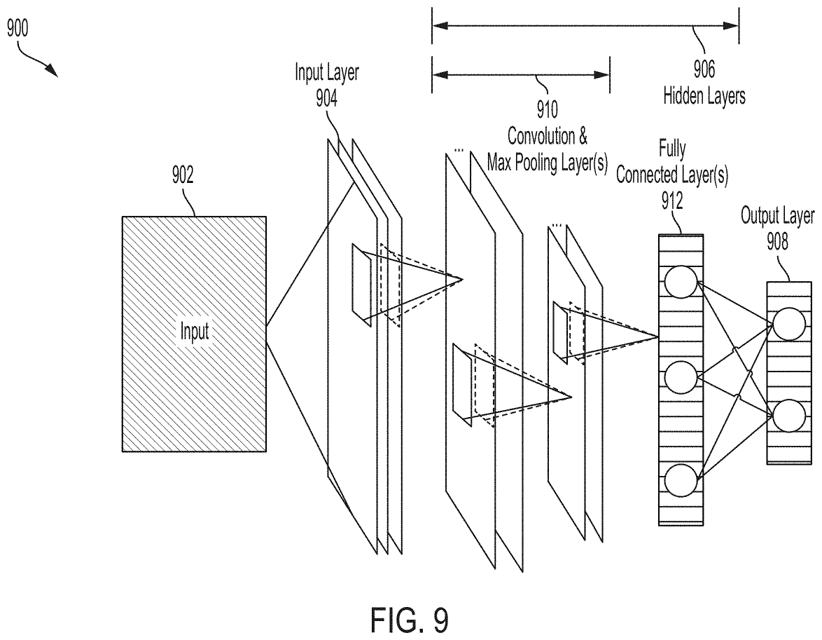

FIG. 6 shows an overview of an illustrative algorithm 600 for generating a machine learning model to be used in treating a neurological disorder, in accordance with some embodiments of the technology described herein. The inputs to the model include the patient specific pre-collected MR and/or CT data, sonication protocol, and/or transducers' configuration (e.g., spatial arrangement), as well as material properties such as mechanical and electrical properties, e.g., speed of sounds, density, elasticity, etc. After some computer-processing, these inputs may be fed into a physical acoustics model (such as linear/nonlinear acoustics, electrodynamics, nonlinear continuum, etc.). In FIG. 6, nodes A and B represent the outputs of the physical acoustics model and the acquired data, which could be in several forms, including but not limited to the frequency response, impulse/transient response, or distribution of acoustic modes. Both A and B are fed into a machine learning model, e.g., a deep neural network, a convolutional neural network as described with respect to FIG. 9, or another suitable machine learning model. The final output can be the frequency, amplitude, the acoustic beam profile, and other requirements, such as expected temperature elevation and/or radiation force. The machine learning model may be trained and implemented in real-time or near real-time. For the transducer array including multiple elements, in the implementation phase, the model may be executed several times by manipulating the pulse amplitude, phase, frequency, and/or time duration of each transducer element. The feedback may include the beam profile given by the model or determined based on an output of the model. Based on the feedback of the model, the pulse amplitude, phase, frequency, and/or time duration may be adjusted iteratively to achieve a desired focusing performance, e.g., a tight focus with a desired beam width and/or energy level. The calculated parameters may then be fed into the device, e.g., device 200, to perform the neuro-modulation in the subject. For example, based on the output of the machine learning model, an instruction may be generated to transmit to the brain of the patient the ultrasound radiation. At a subsequent time, the output of the physical acoustics model and updated data acquired from the brain of the patient may be fed into the machine learning model and an updated instruction may be generated to transmit to the brain of the patient the ultrasound radiation.

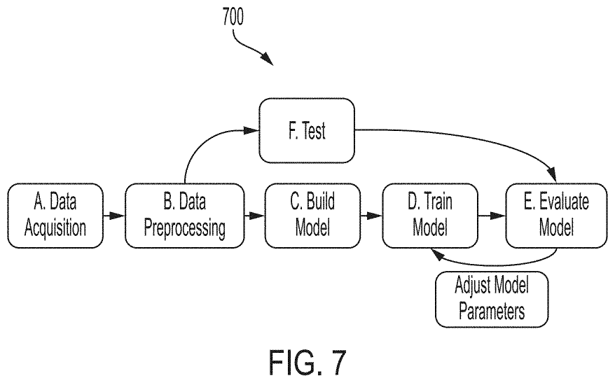

Exemplary steps 700 often undertaken to construct and deploy the algorithms described herein are shown in FIG. 7, including data acquisition, data preprocessing, building a model, training the model, evaluating the model, testing, and adjusting model parameters.

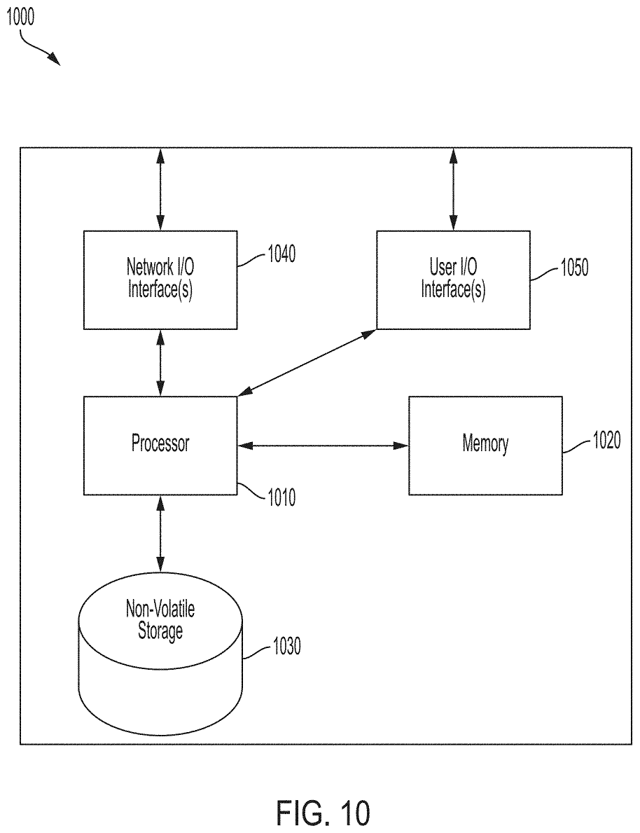

FIG. 8 shows an illustrative flow diagram 800 for guiding ultrasound radiation using a device to treat a neurological disorder, e.g., the device as described with respect to FIGS. 1-3, in accordance with some embodiments of the technology described herein. This process may be implemented on a processor, e.g., as described with respect to FIG. 10, and may be included in the device or a hub, separate from the device, such as a watch or a smart phone.

At step 802, the processor may receive as a first input patient scan data. The patient scan data may include patient specific pre-collected MR and/or CT data.

At step 804, the processor may receive as a second input information regarding configuration and/or properties of one or more ultrasound transmitters adapted to transmit to the brain the ultrasound radiation. For example, the configuration may include spatial arrangement of ultrasound transmitters, and the properties may include at least one of sound signal speeds, elasticity, and/or density.

At step 806, the processor may process at least one of the first and second inputs and feeding the processed at least one of the first and second inputs into a physical acoustics model. The physical acoustics model may employ at least one of linear acoustics, non-linear acoustics, electrodynamics, and/or non-linear continuums.

At step 808, based on an output of the physical acoustics model and acquired data from the brain of the patient, the processor may generate an instruction to transmit to the brain of the patient the ultrasound radiation. The acquired data from the brain of the patient may include at least one of a frequency response, an impulse/transient response and/or a distribution of acoustic modes.

In some embodiments, the processor may feed the output of the physical acoustics model and the acquired data from the brain of the patient into a machine learning model. The machine learning model may include a deep neural network, a convolutional neural network as described with respect to FIG. 9, or another suitable machine learning model.

Based on an output of the machine learning model, the processor may generate the instruction to transmit to the brain of the patient the ultrasound radiation. The output of the machine learning model may include at least one of frequency, amplitude, acoustic beam profile, temperature elevation or reduction, and/or radiation force.

Additionally or alternatively, the output of the physical acoustics model and updated data acquired from the brain of the patient may be fed into the machine learning model and an updated instruction may be generated to transmit to the brain of the patient the ultrasound radiation, e.g., ultrasound radiation having a different intensity, another region of the brain, and/or another suitable update to treat the patient's neurological disorder.

FIG. 9 shows a convolutional neural network 900 that may be used in treating a neurological disorder, in accordance with some embodiments of the technology described herein. The statistical or machine learning model described herein may include the convolutional neural network 900, and additionally or alternatively another type of network, suitable for predicting frequency, amplitude, the acoustic beam profile, and other requirements, such as expected temperature elevation and/or radiation force, etc. As shown, the convolutional neural network comprises an input layer 904 configured to receive information about the input 902 (e.g., a tensor), an output layer 908 configured to provide the output (e.g., classifications in an n-dimensional representation space), and a plurality of hidden layers 906 connected between the input layer 904 and the output layer 908. The plurality of hidden layers 906 include convolution and pooling layers 910 and fully connected layers 912.

The input layer 904 may be followed by one or more convolution and pooling layers 910. A convolutional layer may comprise a set of filters that are spatially smaller (e.g., have a smaller width and/or height) than the input to the convolutional layer (e.g., the input 902). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 910 may be followed by fully connected layers 912. The fully connected layers 912 may comprise one or more layers each with one or more neurons that receives an input from a previous layer (e.g., a convolutional or pooling layer) and provides an output to a subsequent layer (e.g., the output layer 908). The fully connected layers 912 may be described as “dense” because each of the neurons in a given layer may receive an input from each neuron in a previous layer and provide an output to each neuron in a subsequent layer. The fully connected layers 912 may be followed by an output layer 908 that provides the output of the convolutional neural network. The output may be, for example, an indication of which class, from a set of classes, the input 902 (or any portion of the input 902) belongs to. The convolutional neural network may be trained using a stochastic gradient descent type algorithm or another suitable algorithm. The convolutional neural network may continue to be trained until the accuracy on a validation set (e.g., a held out portion from the training data) saturates or using any other suitable criterion or criteria.

It should be appreciated that the convolutional neural network shown in FIG. 9 is only one example implementation and that other implementations may be employed. For example, one or more layers may be added to or removed from the convolutional neural network shown in FIG. 9. Additional example layers that may be added to the convolutional neural network include: a pad layer, a concatenate layer, and an upscale layer. An upscale layer may be configured to upsample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output. As another example, in some embodiments, one or more convolutional, transpose convolutional, pooling, unpooling layers, and/or batch normalization may be included in the convolutional neural network. As yet another example, the architecture may include one or more layers to perform a nonlinear transformation between pairs of adjacent layers. The non-linear transformation may be a rectified linear unit (ReLU) transformation, a sigmoid, and/or any other suitable type of non-linear transformation, as aspects of the technology described herein are not limited in this respect.

Any suitable optimization technique may be used for estimating neural network parameters from training data. For example, one or more of the following optimization techniques may be used: stochastic gradient descent (SGD), mini-batch gradient descent, momentum SGD, Nesterov accelerated gradient, Adagrad, Adadelta, RMSprop, Adaptive Moment Estimation (Adam), AdaMax, Nesterov-accelerated Adaptive Moment Estimation (Nadam), AMSGrad.

Convolutional neural networks may be employed to perform any of a variety of functions described herein. It should be appreciated that more than one convolutional neural network may be employed to make predictions in some embodiments.

### Epilepsy and Seizure

In some embodiments, the described device and methods may be used to treat epilepsy, which is a group of neurological disorders characterized by epileptic seizures. Epileptic seizures are episodes that can vary from brief and nearly undetectable periods to long periods of vigorous shaking. These episodes can result in physical injuries, including occasionally broken bones. In epilepsy, seizures tend to recur and have no immediate underlying cause.

The cause of most cases of epilepsy is unknown. Some cases occur as the result of brain injury, stroke, brain tumors, infections of the brain, and birth defects through a process known as epileptogenesis. Epileptic seizures are the result of excessive and abnormal neuronal activity in the cortex of the brain. The diagnosis involves ruling out other conditions that might cause similar symptoms, such as fainting, and determining if another cause of seizures is present, such as alcohol withdrawal or electrolyte problems. This may be partly done by imaging the brain and performing blood tests. Epilepsy can often be confirmed with an electroencephalogram (EEG), described further below.

As of 2015, about 39 million people have epilepsy. Nearly 80% of cases occur in the developing world. In 2015, it resulted in 125,000 deaths up from 112,000 deaths in 1990. Epilepsy is more common in older people. In the developed world, onset of new cases occurs most frequently in babies and the elderly. In the developing world, onset is more common in older children and young adults, due to differences in the frequency of the underlying causes. About 5-10% of people will have an unprovoked seizure by the age of 80, and the chance of experiencing a second seizure is between 40 and 50%. In many areas of the world, those with epilepsy either have restrictions placed on their ability to drive or are not permitted to drive until they are free of seizures for a specific length of time.

The diagnosis of epilepsy is typically made based on observation of the seizure onset and the underlying cause. An electroencephalogram (EEG) to look for abnormal patterns of brain waves and neuroimaging (CT scan or MRI) to look at the structure of the brain are also usually part of the workup. While figuring out a specific epileptic syndrome is often attempted, it is not always possible. Video and EEG monitoring may be useful in difficult cases.

An electroencephalogram (EEG) can assist in showing brain activity suggestive of an increased risk of seizures. It is only recommended for those who are likely to have had an epileptic seizure on the basis of symptoms. In the diagnosis of epilepsy, electroencephalography may help distinguish the type of seizure or syndrome present.

Diagnostic imaging by CT scan and MRI is recommended after a first non-febrile seizure to detect structural problems in and around the brain. MRI is generally a better imaging test except when bleeding is suspected, for which CT is more sensitive and more easily available. If someone attends the emergency room with a seizure but returns to normal quickly, imaging tests may be done at a later point.

Wristbands or bracelets denoting their condition are occasionally worn by epileptics should they need medical assistance. Epilepsy is usually treated with daily medication once a second seizure has occurred, while medication may be started after the first seizure in those at high risk for subsequent seizures. Diet, alternative medicine, and people's self-management of their condition (such as avoidance therapy consisting of minimizing or eliminating triggers) may be useful. In drug-resistant cases or cases experiencing severe side effects different and harsher management options may be considered including the implantation of a neurostimulator or neurosurgery.

Epilepsy surgery may be an option for people with focal seizures that remain a problem despite other treatments. These other treatments include at least a trial of two or three medications. The goal of surgery is total control of seizures and this may be achieved in 60-70% of cases. Common procedures include cutting out the hippocampus via an anterior temporal lobe resection, removal of tumors, and removing parts of the neocortex. Some procedures such as a corpus callosotomy are attempted in an effort to decrease the number of seizures rather than cure the condition. Following surgery, medications may be slowly withdrawn in many cases.

Neurostimulation may be another option in those who are not candidates for surgery. Three types have been shown to be effective in those who do not respond to medications: vagus nerve stimulation, anterior thalamic stimulation, and closed-loop responsive stimulation.

Epilepsy cannot usually be cured, unless surgery is performed. However, the outcome of surgery can lead to unexpected harsh outcomes such as loss of functionality of certain abilities such as speech, control over movements, etc. In the developing world, 75% of people are either untreated or not appropriately treated. In Africa, 90% do not get treatment. This is partly related to appropriate medications not being available or being too expensive.

People with epilepsy are at an increased risk of death. This increase is between 1.6 and 4.1-fold greater than that of the general population and is often related to: the underlying cause of the seizures, status epilepticus, suicide, trauma, and sudden unexpected death in epilepsy (SUDEP). Death from status epilepticus is primarily due to an underlying problem rather than missing doses of medications. The risk of suicide is between two and six times higher in those with epilepsy. The cause of this is unclear. The greatest increase in mortality from epilepsy is among the elderly. Those with epilepsy due to an unknown cause have little increased risk. In the developing world, many deaths are due to untreated epilepsy leading to falls or status epilepticus.

### Electroencephalography

Electroencephalography (EEG) is an electrophysiological monitoring method to record electrical activity of the brain. It is typically noninvasive, with the electrodes placed along the scalp, although invasive electrodes are sometimes used such as in electrocorticography. EEG measures voltage fluctuations resulting from ionic current within the neurons of the brain. EEG is most often used to diagnose epilepsy, which causes abnormalities in EEG readings.

EEG may have a poor spatial resolution. Often for proper diagnosis or detection of epilepsy both high temporal resolution and spatial resolution are required. Functional magnetic resonance imaging (MRI) and computed tomography (CT) can be used for detection of epileptic events. They may provide better spatial resolution. However, they may have poor temporal resolution. Moreover, they may be expensive and may not portable. Despite limited spatial resolution, EEG may be a valuable tool for research and diagnosis as one of few mobile techniques available and offering millisecond-range temporal resolution which may not be possible with CT, PET or MRI.

### Example Computer Architecture

An illustrative implementation of a computer system 1000 that may be used in connection with any of the embodiments of the technology described herein is shown in FIG. 10. The computer system 1000 includes one or more processors 1010 and one or more articles of manufacture that comprise non-transitory computer-readable storage media (e.g., memory 1020 and one or more non-volatile storage media 1030). The processor 1010 may control writing data to and reading data from the memory 1020 and the non-volatile storage device 1030 in any suitable manner, as the aspects of the technology described herein are not limited in this respect. To perform any of the functionality described herein, the processor 1010 may execute one or more processor-executable instructions stored in one or more non-transitory computer-readable storage media (e.g., the memory 1020), which may serve as non-transitory computer-readable storage media storing processor-executable instructions for execution by the processor 1010.

Computing device 1000 may also include a network input/output (I/O) interface 1040 via which the computing device may communicate with other computing devices (e.g., over a network), and may also include one or more user I/O interfaces 1050, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The embodiments described herein can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described herein can be generically considered as one or more controllers that control the functions discussed herein. The one or more controllers can be implemented in numerous ways, such as with dedicated hardware, or with general purpose hardware (e.g., one or more processors) that is programmed using microcode or software to perform the functions recited herein.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the functions discussed herein of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the functions discussed herein, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The terms “program” or “software” are used herein in a generic sense to refer to any type of computer code or set of processor-executable instructions that can be employed to program a computer or other processor to implement various aspects of embodiments as discussed herein. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing,” “involving,” and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

While some aspects and/or embodiments described herein are described with respect to epilepsy-related applications, these aspects and/or embodiments may be equally applicable to monitoring and/or treating symptoms for any suitable neurological disorder or brain condition. Any limitations of the embodiments described herein are limitations only of those embodiments, and are not limitations of any other embodiments described herein.

What is claimed is:

1. A device comprising:
a substrate; and
at least one capacitive micromachined ultrasonic transducer (CMUT) located on or in the substrate that provides ultrasound radiation to a brain of a patient.

2. The device as claimed in claim 1 wherein the substrate is flexible.

3. The device as claimed in claim 2 wherein the substrate is made from a printed circuit board (PCB).

4. The device as claimed in claim 1 wherein the at least one CMUT includes an array of a plurality of CMUTs.

5. The device as claimed in claim 1 wherein the substrate is embedded in or on a cap intended to be worn on a scalp of the patient.

6. The device as claimed in claim 1 wherein the at least one CMUT is powered and/or driven wirelessly.

7. The device as claimed in claim 1 wherein the ultrasound radiation is guided within the brain through a computer implemented simulation model.

8. The device as claimed in claim 7 wherein the computer implemented simulation model includes a machine learning model.

9. The device as claimed in claim 7 wherein the computer implemented simulation model includes as an input a scan of the brain of the patient.

10. The device as claimed in claim 1 wherein the ultrasound radiation is guided within the brain of the patient through magnetic resonance imaging (MRI) monitoring.

11. A wearable or implantable device for disposal on a scalp of a patient comprising:
a substrate; and
at least one capacitive micromachined ultrasonic transducer (CMUT) located on or in the substrate that provides ultrasound radiation to a brain of the patient.

12. A method of guiding ultrasound radiation in the brain of a patient comprising:
receiving as a first input patient scan data;
receiving as a second input information regarding configuration and/or properties of one or more ultrasound transmitters adapted to transmit to the brain the ultrasound radiation;
processing at least one of the first and second inputs and feeding the processed at least one of the first and second inputs into a physical acoustics model; and
based on an output of the physical acoustics model and acquired data from the brain of the patient, generating an instruction to transmit to the brain of the patient the ultrasound radiation.

13. The method of claim 12 further comprising:
feeding the output of the physical acoustics model and the acquired data from the brain of the patient into a machine learning model; and
based on an output of the machine learning model, generating the instruction to transmit to the brain of the patient the ultrasound radiation.

14. The method as claimed in claim 12 wherein the configuration includes a spatial arrangement of the one or more ultrasound transmitters.

15. The method as claimed in claim 12 wherein the properties include at least one of sound signal speeds, elasticity, and/or density.

16. The method as claimed in claim 12 wherein the physical acoustics model employs at least one of linear acoustics, non-linear acoustics, electrodynamics, and/or non-linear continuums.

17. The method as claimed in claim 12 wherein the acquired data from the brain of the patient fed into the machine learning model includes at least one of a frequency response, an impulse/transient response, and/or a distribution of acoustic modes.

18. The method as claimed in claim 13 wherein the output of the machine learning model includes at least one of frequency, amplitude, acoustic beam profile, temperature elevation or reduction, and/or radiation force.

19. The method as claimed in claim 13 wherein the machine learning model comprises a convolutional neural network.

20. The method as claimed in claim 19 further including building the machine learning model and/or training with data the machine learning model.

21. The method as claimed in claim 13 further comprising feeding the output of the physical acoustics model and updated data acquired from the brain of the patient into the machine learning model.

22. The method as claimed in claim 21 further comprising generating an updated instruction to transmit to the brain of the patient the ultrasound radiation.
